(19) United States
(12) Patent Application Publication
(10) Pub. No.: US 2025/0339181 A1
(43) Pub. Date: Nov. 6, 2025

(54) Spinal Rods, Sets, Systems, and Methods

(71) Applicant: Craig Black, Florence, SC (US)

(72) Inventor: Craig Black, Florence, SC (US)

(21) Appl. No.: 19/178,248

(22) Filed: Apr. 14, 2025

Related U.S. Application Data
(60) Provisional application No. 63/633,298, filed on Apr. 12, 2024.

(51) Int. Cl. A61B 17/70; A61B 17/56

(52) U.S. Cl. CPC A61B 17/7007; A61B 17/7011; A61B 17/7083; A61B 17/7091; A61B 2017/564

(57) ABSTRACT

Example spinal rods, sets of spinal rods, insertion tools, systems for Implanting spinal rods, methods of implanting spinal rods, methods of making spinal rods, methods of making sets of spinal rods, methods of making Insertion tools, and methods of making systems for implanting spinal rods in a patient are described.

### FIELD

The disclosure relates generally to the field of medical devices. More particularly, the disclosure relates to spinal rods useful in the stabilization of bones in a patient. The disclosure also relates to sets of spinal rods, insertion tools for implanting spinal rods, systems for implanting spinal rods, and methods of implanting spinal rods. The disclosure also relates to methods of making spinal rods, methods of making sets of spinal rods, methods of making insertion tools, and methods of making systems for implanting a spinal rod in a patient.

### BACKGROUND

A need exists for new and improved spinal rods, sets of spinal rods, insertion tools, systems for implanting spinal rods, methods of implanting spinal rods, methods of making spinal rods, methods of making sets of spinal rods, methods of making insertion tools, and methods of making systems for implanting spinal rods in a patient.

### BRIEF SUMMARY OF SELECTED EXAMPLES

Various spinal rods, sets of spinal rods, insertion tools, systems for implanting spinal rods, methods of implanting spinal rods, methods of making spinal rods, methods of making sets of spinal rods, methods of making insertion tools, and methods of making systems for implanting spinal rods in a patient are described herein.

Additional understanding of these example spinal rods, sets of spinal rods, insertion tools, systems for implanting spinal rods, methods of implanting spinal rods, methods of making spinal rods, methods of making sets of spinal rods, methods of making insertion tools, and methods of making systems for implanting spinal rods in a patient can be obtained by review of the detailed description of selected examples, below, and the referenced drawings.

### BRIEF DESCRIPTION OF FIGURES

FIG. 1 is a perspective view of an example spinal rod. The spinal rod is illustrated with screws used to secure the spinal rod to vertebrae of a patient.

FIG. 2 is a side view of the spinal rod and screws illustrated in FIG. 1.

FIG. 3 is a top view of the spinal rod and screws illustrated in FIG. 1.

FIG. 3A is a sectional view of the spinal rod illustrated in FIG. 1, taken along line 3A-3A in FIG. 3.

FIG. 3B is a sectional view of the spinal rod illustrated in FIG. 1, taken along line 3B-3B in FIG. 3.

FIG. 3C is a sectional view of an alternative spinal rod.

FIG. 3D is a sectional view of an alternative spinal rod.

FIG. 3E is a sectional view of an alternative spinal rod.

FIG. 3F is a sectional view of an alternative spinal rod.

FIG. 4 is a perspective view of another example spinal rod. The spinal rod is illustrated with screws used to secure the spinal rod to vertebrae of a patient.

FIG. 5 is a side view of the spinal rod and screws illustrated in FIG. 4.

FIG. 6 is a top view of the spinal rod and screws illustrated in FIG. 4.

FIG. 7 is a perspective view of another example spinal rod. The spinal rod is illustrated with screws used to secure the spinal rod to vertebrae of a patient.

FIG. 8 is a side view of the spinal rod and screws illustrated in FIG. 7.

FIG. 9 is a top view of the spinal rod and screws illustrated in FIG. 7.

FIG. 10 is a perspective view of an example set of spinal rods. Each spinal rod in the set is illustrated with screws used to secure the spinal rod to vertebrae of a patient.

FIG. 11 is a perspective view of a prior art insertion tool for implanting spinal rods.

FIG. 12 is a perspective view of an example insertion tool for implanting a spinal rod.

FIG. 13 is a perspective view of another example insertion tool for implanting a spinal rod.

FIG. 14 is a perspective view of an example system for implanting a spinal rod.

FIG. 15 is a perspective view of an example system for implanting spinal rods.

FIG. 16 is a flowchart illustration of an example method of implanting a spinal rod.

FIG. 17 is a flowchart illustration of an example method of implanting spinal rods.

### DETAILED DESCRIPTION OF SELECTED EXAMPLES

The following detailed description and the appended drawings describe and illustrate various example spinal rods, sets of spinal rods, insertion tools, systems for implanting spinal rods, methods of implanting spinal rods, methods of making spinal rods, methods of making sets of spinal rods, methods of making insertion tools, and methods of making systems for implanting spinal rods in a patient. The description and illustration of these examples are provided to enable one skilled in the art to make and use example spinal rods, sets of spinal rods, insertion tools, and systems for implanting spinal rods, and to practice example methods of implanting spinal rods, methods of making spinal rods, methods of making sets of spinal rods, methods of making insertion tools, and methods of making systems for implanting spinal rods in a patient. The inclusion of detailed descriptions of these examples is not intended to limit the scope of the invention, or its protection, in any manner. The invention is capable of being practiced or carried out in various ways and the examples described and illustrated herein are not considered exhaustive.

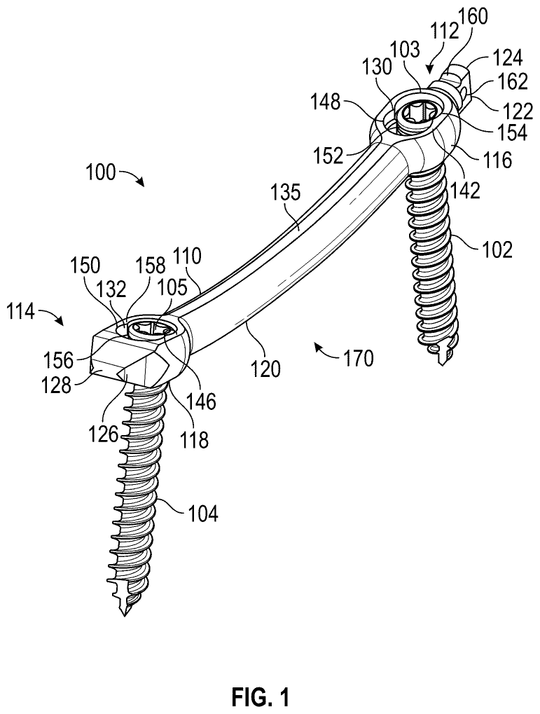

Each of FIGS. 1, 2, 3, 3A and 3B illustrates an example spinal rod 100. In each of FIGS. 1, 2, and 3, the spinal rod 100 is illustrated with screws 102, 104 used to secure the spinal rod 100 to vertebrae of a patient. Spinal rod 100 is an elongate member 110 having a proximal end 112 and a distal end 114. The elongate member 110 defines a proximal anchor portion 116 distal to the proximal end 112, a distal anchor portion 118 proximal to the distal end 114, and a body 120 extending between the proximal anchor portion 116 and the distal anchor portion 118. The proximal end 112 defines an engagement extension 122 proximal to the proximal anchor portion 116 and that defines the terminal proximal surface 124 of the elongate member 110. The distal end 114 defines a nose 126 distal to the distal anchor portion 118 and that defines the terminal distal surface 128 of the elongate member 110. The proximal anchor portion 116 defines a passageway 130 through which an orthopedic screw, such as screw 102, can be disposed for securing spinal rod 100 to vertebrae of a patient. Similarly, distal anchor portion 118 defines a passageway 132 through which an orthopedic screw, such as screw 104, can be disposed for securing spinal rod 100 to vertebrae of a patient.

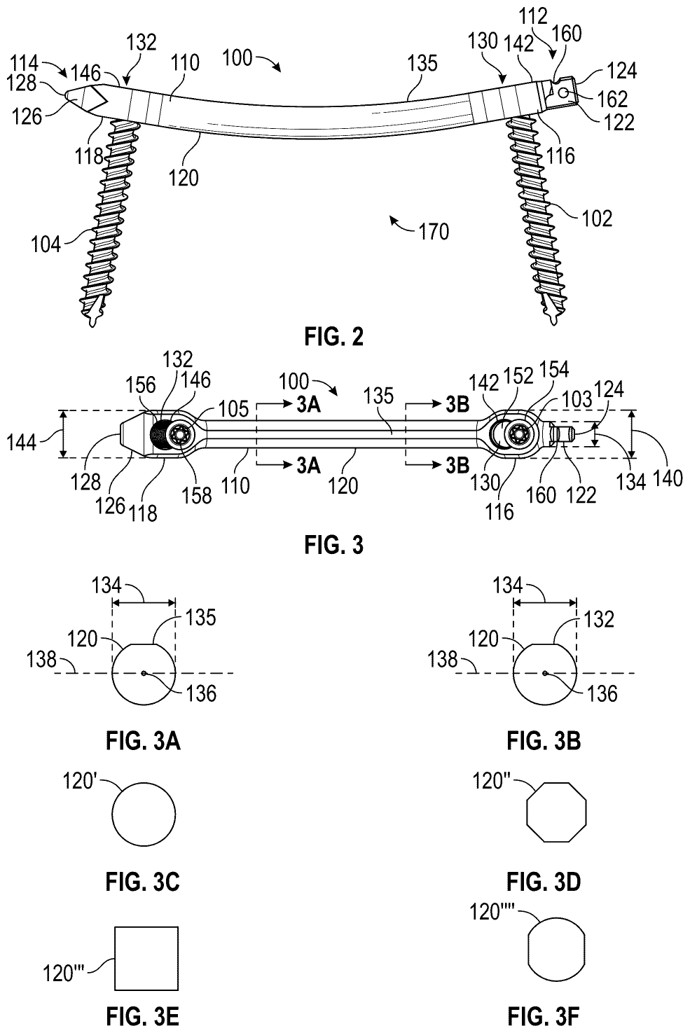

As best illustrated in FIGS. 1 and 2, elongate member 110 defines a curve 170 along its axial length.

Body 120 has a substantially circular cross-sectional shape along its axial length, extending between the proximal anchor portion 116 and the distal anchor portion 118. As best illustrated in FIGS. 3, 3A and 3B, body 120 includes a flat surface 135 on the top of the body 120 as illustrated and positioned in FIGS. 1, 2, and 3. Flat surface 135 extends the entire axial length between the proximal anchor portion 116 and the distal anchor portion 118. While considered optional, inclusion of flat surface 135 is considered advantageous at least because it provides tactile feedback regarding orientation of spinal rod 100 to an individual handling spinal rod 100, such as a clinician during implantation of spinal rod 100 in a patient. As best illustrated in FIGS. 3A and 3B, body 120 has a width 134 measured through the geometric center 136 of a cross-section of body 120 and extending on an axis 138 that is parallel to a plane that includes flat surface 135.

Each of FIGS. 3C, 3D, 3E, and 3F illustrates an alternative body having a different cross-sectional shape than that of body 120. FIG. 3C illustrates an alternative body 120′ having a circular cross-sectional shape, with no flat surface on any portion of the circumference. FIG. 3D illustrates an alternative body 120″ having an octagon cross-sectional shape, with eight flat surfaces on the circumference. FIG. 3E illustrates an alternative body 120′″ having a square cross-sectional shape, with four flat surfaces on the circumference. FIG. 3F illustrates an alternative body 120′″ having a flat circle cross-sectional shape, with two opposing flat surfaces on the circumference. A spinal rod according to an embodiment can include different cross-sectional shapes at different axial positions along the length of the body. For example, a spinal rod according to an embodiment can have a circular or substantially circular cross-sectional shape at a location adjacent the proximal anchor portion and a polygonal cross-sectional shape at a location adjacent the distal anchor portion. In one embodiment, a spinal rod has a flat circle cross-sectional shape, such as the cross-sectional shape illustrated in FIG. 3F, at a location adjacent the proximal anchor portion and a circular or substantially circular cross-sectional shape at a location adjacent the distal anchor portion. It is noted, too, that a spinal rod according to an embodiment can have more than two different cross-sectional shapes along the length of its body. A skilled artisan will be able to determine if it is desirable to include multiple, different cross-sectional shapes along the axial length of the body and the specific cross-sectional shapes or cross-sectional shapes at specific positions along the axial length of the body based on various considerations, including any desired localized rigidity of body based on data or other considerations related to the specific patient into which the particular spinal rod is to be implanted. For example, a spinal rod according to an embodiment can include multiple, different cross-sectional shapes along the axial length of the body of the spinal rod to provide multiple, different localized elastic moduli, each of which can be based on localized information for the specific location in the implant site at which that portion of the body will be positioned within the implant site in a specific patient. For example, based on pre-operative imaging of a patient into which a spinal rod is to be implanted, it may be desirable to have a higher localized rigidity in the lengthwise center of body than at the ends of the body near the anchor portions. This specific spinal rod, then, could be manufactured to have circular or substantially circular cross-sectional shapes at the body near the proximal and distal anchor portions, and a polygonal cross-sectional shape at the mid-point along the axial length of the body, between the axial portions having circular or substantially circular cross-sectional shapes. For the portion having a polygonal cross-sectional shape, any desirable cross-sectional shape could be used and a skilled artisan will be able to select a suitable cross-sectional shape based on various considerations, including the desired localized rigidity, any desired tactile considerations, and other considerations. Examples of suitable polygonal cross-sectional shapes include, but are not limited to, a triangular cross-sectional shape, a square cross-sectional shape, such as that illustrated in FIG. 3E, a rectangular cross-sectional shape, a hexagonal cross-sectional shape, an octagonal cross-sectional shape, such as that illustrated in FIG. 3D, and other polygonal cross-sectional shapes. Also, in this specific example, if the body includes circular or substantially circular cross-sectional shapes near the proximal and distal anchor portions, the body can have a flat circle cross-sectional shape, such as that illustrated in FIG. 3F, at the mid-point along the axial length of the body, between the axial portions having circular or substantially circular cross-sectional shapes.

Each of proximal anchor portion 116 and distal anchor portion 118 is an enlarged portion of the elongate member 110 relative to body 120. That is, proximal anchor portion 116 has a width 140, measured through the geometric center of a cross-section of the proximal anchor portion 116 and extending on an axis that is parallel to a plane that includes flat surface 142 of proximal anchor portion 116. Similarly, distal anchor portion 118 has a width 144, measured through the geometric center of a cross-section of the distal anchor portion 118 and extending on an axis that is parallel to a plane that includes flat surface 146 of distal anchor portion 118.

Each of passageways 130, 132 extends entirely through elongate member 110, allowing a screw, such as one of screws 102, 104, to be disposed in and extend through a respective passageway 130, 132. As best illustrated in FIGS. 1 and 3, elongate member 110 advantageously defines a shoulder 148 that extends around passageway 130 to provide a mechanical stop for a head portion 103 of screw 102 disposed in passageway 130. Similarly, elongate member 110 advantageously defines a shoulder 150 that extends around passageway 132 to provide a mechanical stop for a head portion 105 of screw 104 disposed in passageway 132.

In this embodiment, elongate member 110 defines first 152 and second 154 scallops that form passageway 130. Each scallop 152, 154 is sized and configured to receive a screw, such as screw 102, providing two distinct axial paths along which screw 102 can extend through passageway 130 and proximal anchor portion 116. Similarly, elongate member 110 defines first 156 and second 158 scallops that form passageway 130. Each scallop 156, 158 is sized and configured to receive a screw, such as screw 104, providing two distinct axial paths along which screw 104 can extend through passageway 132 and distal anchor portion 118. As described in more detail below, one or more of passageways 130, 132 can define an axial trajectory for a screw, such as one of screws 102, 104, that is based upon one or more attributes of the specific individual patient into which spinal rod 100 is intended to be implanted, such that passageways 130, 132 guide screws 102, 104 into a specific location in vertebrae adjacent the implant site for anchoring the spinal rod 100.

Engagement extension 122 defines structure for engagement with a insertion tool for implanting the spinal rod 100 within an implant site in a patient. In the illustrated example, engagement extension 122 is a polygonal projection defining a notch 160 and an opening 162 that facilitate engagement with a insertion tool. Also, as described in more detail below, engagement extension 122 extends away from the proximal anchor portion 116 at an angle that is based upon the specific implant site of the patient into which the spinal rod is to be implanted. For example, the engagement extension 122 extends away from proximal anchor portion 116 at an angle that, when attached to a insertion tool, places spinal rod 100 in a spatial orientation suitable for advancing the spinal rod 100 into the specific implant site of the specific patient for whom spinal rod 100 was designed and manufactured. In the illustrated embodiment, in which spinal rod 100 defines curve 170 extending along the axial length of the elongate member 110, engagement extension 120 is continuous with the curve 170. In other embodiments, engagement extension can extend away from a longitudinal axis of elongate member at any suitable angle based upon one or more attributes of the implant site into which the spinal rod is intended to be implanted. Examples of suitable angles include, but are not limited to, between about 1° and about 60°, between about 1° and about 45°, between about 1° and about 30°, between about 1° and about 15°, between about 1° and about 10°, between about 1° and about 9°, between about 1° and about 8°, between about 1° and about 7°, between about 1° and about 6°, between about 1° and about 5°, between about 1° and about 4°, between about 1° and about 3°, between about 1° and about 2°, and about 1°.

Nose 126 provides a tapered end to spinal rod 100, which facilitates advancement of spinal rod through tissue during implantation. This structural feature of spinal rod 100 provides an adaptation that makes spinal rod 100 suitable for implantation by open procedure, i.e., surgical procedures, and percutaneous procedures, i.e., minimally invasive procedures.

Spinal rod 100 is includes one or more structural features that are based upon attributes of the specific individual patient into which spinal rod 100 is intended to be implanted, such as attributes of the implant site of the patient into which the spinal rod is intended to be implanted. As such, spinal rod 100 is a patient-specific spinal rod. Examples of structural features that can be based upon attributes of a specific patient include, but are not limited to, pre-operative imaging of the patient, pre-operative imaging of the implant site of the patent, dimensions calculated based on pre-operative imaging of the implant site of the patient, such as distances between bones, anatomical features, or other dimensions, body mass index (BMI) or the patient, and others attributes of the patient and/or the implant site. Examples of structural features of the spinal rod that can be based on attributes of the patient include, but are not limited to, the presence or absence of a curve along the length of the elongate member, the specific curve along the length of the elongate member, the cross-sectional shape of the body, the inclusion of multiple, different cross-sectional shapes along the axial length of the body, the specific, multiple different cross-sectional shapes along the axial length of the body, the width of the body, the inclusion of multiple different widths along the axial length of the body, specific, multiple different widths along the axial length of the body, the outer diameter of the body, the inclusion of multiple different outer diameters along the axial length of the body, specific, multiple different outer diameters along the axial length of the body, the axial trajectory of the passageways through one or both of the proximal anchor portion and the distal anchor portion, which can be the same or different, the angle at which the engagement extension extends away from the proximal anchor portion, and the dimensions of the nose, including the width of the nose and the angle of one or more tapered surfaces on the nose relative to a longitudinal axis of the elongate member. In the illustrated example, the first scallop 152 in the proximal anchor portion 116 defines an axial trajectory for screw 102 that is based upon pre-operative imaging of the implant site in a specific patient. Also, the first scallop 156 in the distal anchor portion 118 defines an axial trajectory for screw 104 that is based upon pre-operative imaging of the implant site in the specific patient. Also, the curve 170 is based upon re-operative imaging of the implant site in the specific patient. Taken together, structural features of spinal rod 100 that are based on patient-specific attributes of the specific patient provides spinal rod 100 with structure that matches the environment into which the spinal rod 100 will be implanted, the implant site, and that provides passageways 130, 132 that guide screws 102, 104 into a specific location in vertebrae adjacent the implant site for anchoring the spinal rod 100.

Spinal rods according to some embodiments include multiple different outer diameters along the axial length of the body of the spinal rod.

Spinal rod 100 can be made using any material suitable for an implantable medical device, including metals, such as stainless steel, titanium, and other metals, and plastics. Further, spinal rod 100 can be made using any suitable fabrication process, including subtractive manufacturing techniques, such as machining, and additive manufacturing techniques, such as 3D printing.

Each of screws 102, 104 is an orthopedic screw adapted for insertion into bone, such as a vertebrae of a patient. Any suitable orthopedic bone screw can be used with spinal rod 100. In this embodiment, screws 102, 104 include head portions 103, 105 that interact with shoulders 148, 150 to seat screws 102, 104 within anchor portions 116, 118 and halt advancement of screws 102, 104 further into bone.

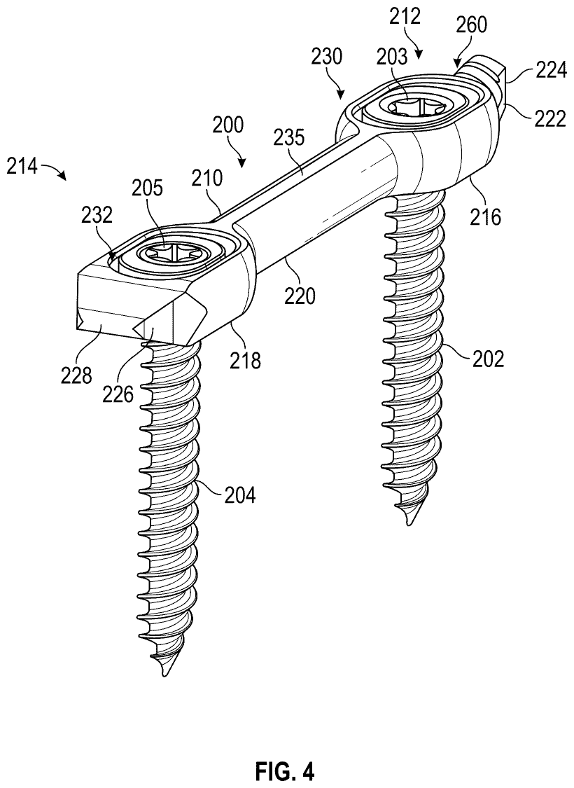

Each of FIGS. 4, 5, and 6 illustrates another example spinal rod 200. The spinal rod 200 is illustrated with screws 202, 204 used to secure the spinal rod 200 to vertebrae of a patient. Spinal rod 200 is similar to spinal rod 100, except as detailed below. This, spinal rod 200 is an elongate member 210 having a proximal end 212 and a distal end 214. The elongate member 210 defines a proximal anchor portion 216 distal to the proximal end 212, a distal anchor portion 218 proximal to the distal end 214, and a body 220 extending between the proximal anchor portion 216 and the distal anchor portion 218. The proximal end 212 defines an engagement extension 222 proximal to the proximal anchor portion 216 and that defines the terminal proximal surface 224 of the elongate member 210. The distal end 214 defines a nose 226 distal to the distal anchor portion 218 and that defines the terminal distal surface 228 of the elongate member 210. The proximal anchor portion 216 defines a passageway 230 through which an orthopedic screw, such as screw 202, can be disposed for securing spinal rod 200 to vertebrae of a patient. Similarly, distal anchor portion 218 defines a passageway 232 through which an orthopedic screw, such as screw 204, can be disposed for securing spinal rod 200 to vertebrae of a patient.

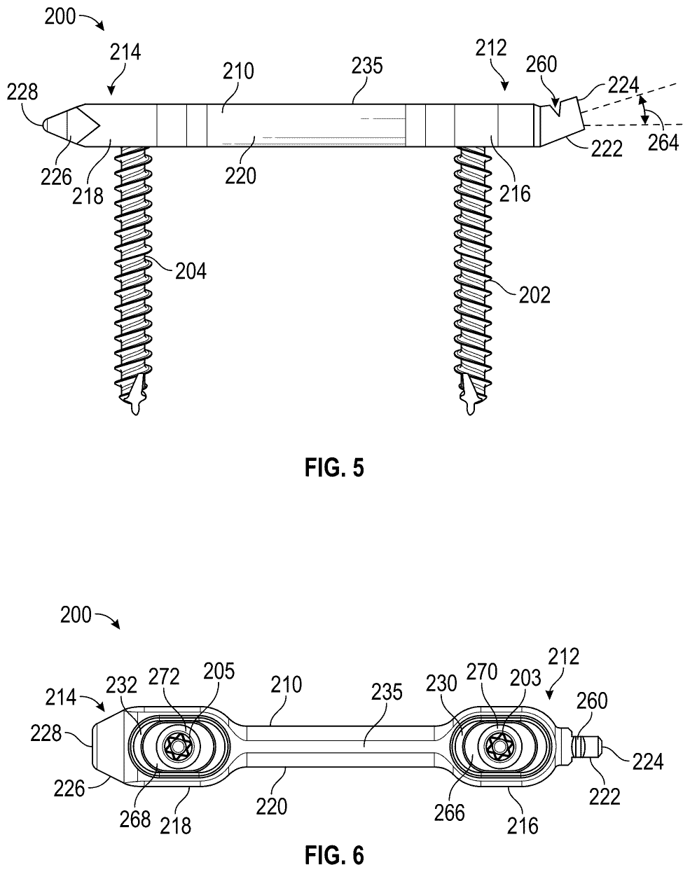

In this embodiment, as best illustrated in FIGS. 4 and 5, elongate member 210 is a linear member, the full length and width of which extends along a single plane. As best illustrated in FIG. 5, engagement extension 222 extends away from proximal anchor portion 216 at an angle 264 of about 30° relative to a longitudinal axis of elongate member 210.

In this embodiment, a first caddy 266 is disposed and held captive within passageway 230 of proximal anchor portion 216. Similarly, a second caddy 268 is disposed and held captive within passageway 232 of distal anchor portion 218. Caddy 266 is a flat-oval-shaped member that defines an inner passageway 270 into which a screw, such as screw 202, can be disposed for anchoring spinal rod 200 to bone. Caddy 266 is slidably movable within passageway 230 of proximal anchor portion 216 along a longitudinal axis of elongate member 210 and rotatable within passageway 230, upward and downward with respect to a longitudinal axis of elongate member 210, by a limited amount of about 45° or less in each direction. Similarly, caddy 268 is a flat-oval-shaped member that defines an inner passageway 272 into which a screw, such as screw 204, can be disposed for anchoring spinal rod 200 to bone. Caddy 268 is slidably movable within passageway 232 of distal anchor portion 218 along a longitudinal axis of elongate member 210 and rotatable within passageway 232, upward and downward with respect to a longitudinal axis of elongate member 210, by a limited amount of about 45° or less in each direction. Inclusion of caddies 266, 268 provides a polyaxial feature to spinal implant 200, allowing for selection of a desired axial trajectory for screws 202, 204 during implantation of spinal rod 200.

While example spinal rod 200 includes first 266 and second 268 caddies, a spinal rod according to an embodiment can have a single caddy in one of a proximal anchor portion and a distal anchor portion, and a different passageway-defining structure in the other of the proximal anchor portion and the distal anchor portion. For example, in one embodiment, a caddy is included in the proximal anchor portion and the distal anchor portion defines first and second scallops, such as those described above and illustrated in FIGS. 1, 2, and 3 with respect to spinal rod 100. As another example, a caddy is included in the proximal anchor portion and the distal anchor portion defines a fixed angle opening for receiving a screw. As another example, a caddy is included in the distal anchor portion and the proximal anchor portion defines first and second scallops, such as those described above and illustrated in FIGS. 1, 2, and 3 with respect to spinal rod 100. As another example, a caddy is included in the distal anchor portion and the proximal anchor portion defines a fixed angle opening for receiving a screw.

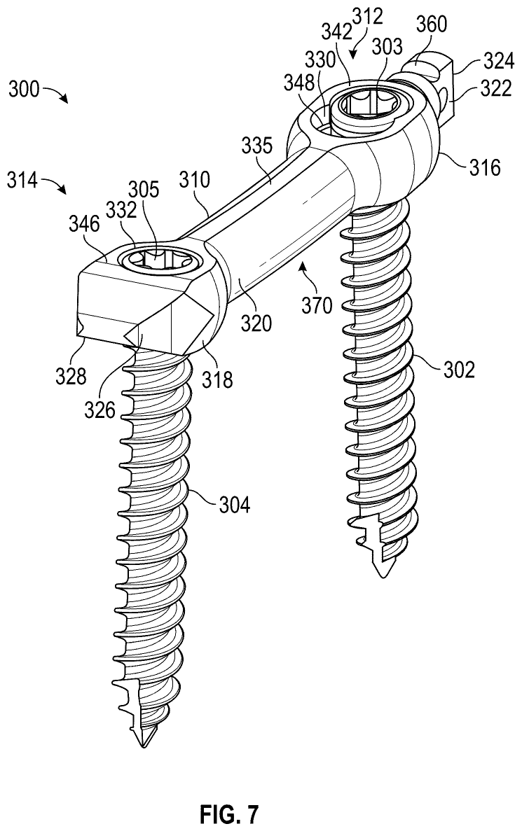

Each of FIGS. 7, 8, and 9 illustrates another example spinal rod 300. The spinal rod 300 is illustrated with screws 302, 304 used to secure the spinal rod 300 to vertebrae of a patient. Spinal rod 300 is similar to spinal rod 100, except as detailed below. This, spinal rod 300 is an elongate member 310 having a proximal end 312 and a distal end 314. The elongate member 310 defines a proximal anchor portion 316 distal to the proximal end 312, a distal anchor portion 318 proximal to the distal end 314, and a body 320 extending between the proximal anchor portion 316 and the distal anchor portion 318. The proximal end 312 defines an engagement extension 322 proximal to the proximal anchor portion 316 and that defines the terminal proximal surface 324 of the elongate member 310. The distal end 314 defines a nose 326 distal to the distal anchor portion 318 and that defines the terminal distal surface 328 of the elongate member 310. The proximal anchor portion 316 defines a passageway 330 through which an orthopedic screw, such as screw 302, can be disposed for securing spinal rod 300 to vertebrae of a patient. Similarly, distal anchor portion 318 defines a passageway 332 through which an orthopedic screw, such as screw 304, can be disposed for securing spinal rod 300 to vertebrae of a patient. As best illustrated in FIG. 7, elongate member 310 advantageously defines a shoulder 348 that extends around passageway 330 to provide a mechanical stop for a head portion 303 of screw 302 disposed in passageway 330.

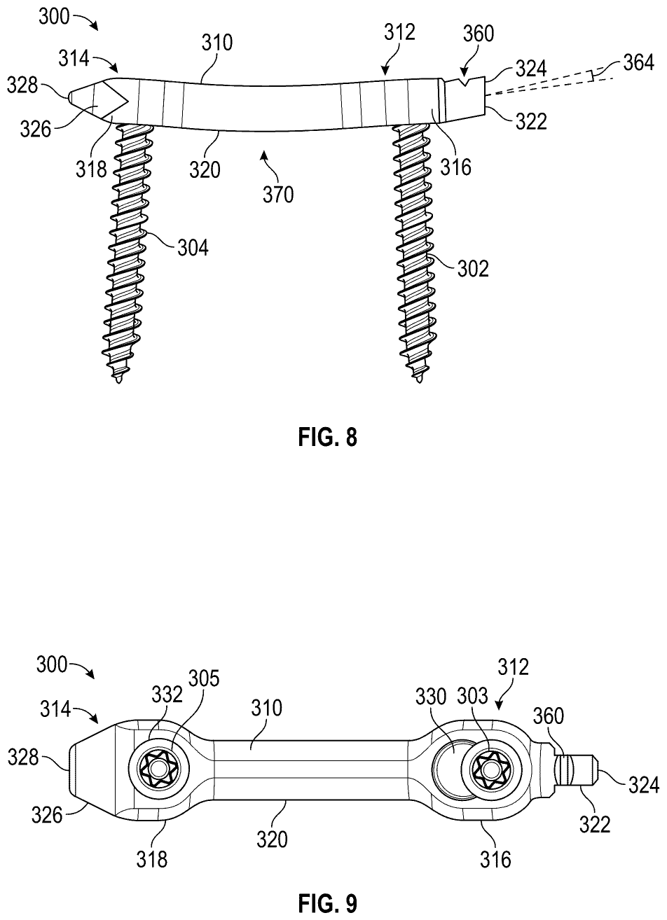

In this embodiment, as best illustrated in FIGS. 7 and 8, elongate member 310 defines curve 370 along its axial length. As best illustrated in FIG. 8, engagement extension 322 extends away from proximal anchor portion 316 at an angle 364 of about 15° relative to a longitudinal axis of elongate member 210.

In this embodiment, elongate member 310 defines first 352 and second 354 scallops that form passageway 330 in proximal anchor portion 316. Each scallop 352, 354 is sized and configured to receive a screw, such as screw 202, providing two distinct axial paths along which screw 202 can extend through passageway 330 and proximal anchor portion 316. Elongate member 110 defines fixed angle passageway 332 in distal anchor portion 318. In this embodiment, each of the first 352 and second 354 scallops and the fixed angle passageway 330 define an axial trajectory for a screw, such as one of screws 302, 304, that is based upon one or more attributes of the specific individual patient into which spinal rod 300 is intended to be implanted, such that passageways 330, 332 guide screws 302, 304 into a specific location in vertebrae adjacent the implant site for anchoring the spinal rod 300.

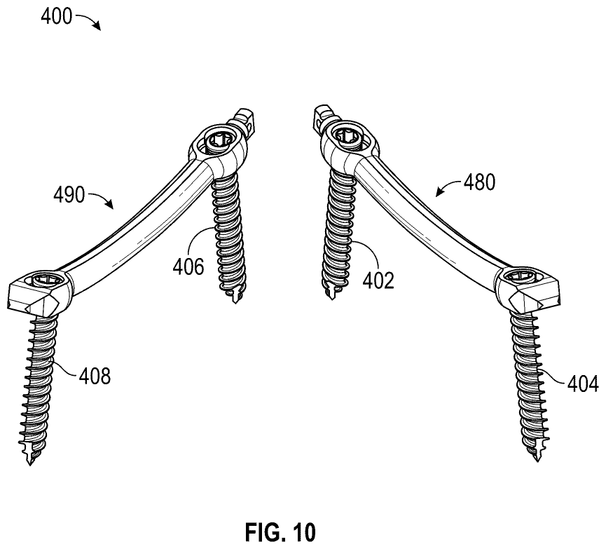

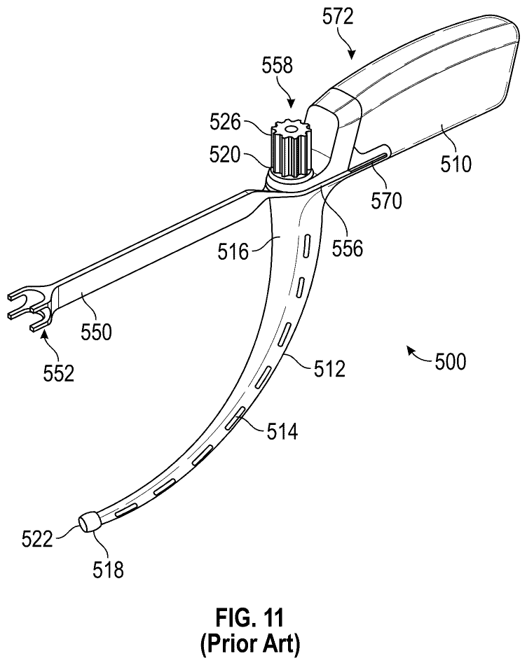

Two or more spinal rods according to one or more embodiments can be grouped together in a set of spinal rods. FIG. 10 illustrates an example set 400 of spinal rods 480, 490. The first spinal rod 480 is illustrated with screws 402, 404 used to secure the spinal rod 480 to vertebrae of a patient. Similarly, the second spinal rod 490 is illustrated with screws 406, 408 used to secure the spinal rod 490 to vertebrae of a patient. In the illustrated example set, the spinal rods 480, 490 are identical. In other embodiments, though one spinal rod in a set of spinal rods can be different than one, more than one, or all other spinal rods in the set of spinal rods, so long as each spinal rod in the set of spinal rods is in accordance with an embodiment. For example, in one embodiment, a set of spinal rods includes a first spinal rod having a first fixed angle passageway in a proximal anchor portion that defines a fixed axial trajectory for a screw that is specific to a first specific implant site in a specific patient, as described above, and a second fixed angle passageway in a distal anchor portion that defines a fixed axial trajectory for a screw that is specific to the first specific implant site in a specific patient, as described above. This example set of spinal rods also includes a second spinal rod having a first fixed angle passageway in a proximal anchor portion that defines a fixed axial trajectory for a screw that is specific to a second specific implant site in the specific patient, as described above, and a second fixed angle passageway in a distal anchor portion that defines a fixed axial trajectory for a screw that is specific to the second specific implant site in a specific patient, as described above. In this example set of spinal rods, each of the first and second spinal rods defines a curve along the longitudinal axis of the elongate member that is based on upon one or more attributes of the respective implant site within the specific patient, such as the spatial positioning of vertebrae within the implant site relative to each other. Also in this example, each of the spinal rods in the set of spinal rods includes an engagement extension that extends away from the proximal anchor portion at an angle to a longitudinal axis of the elongate member that is based upon one or more attributes of the respective implant site within the specific patient, such as the spatial positioning of vertebrae within the implant site relative to each other. Since the first spinal rod defines structure that adapts it for implantation at the first implant site and the second spinal rod defines structure that adapts it for implantation at the second implant site, the respective angles at which the engagement extension of the first spinal rod can extend away from the proximal anchor portion of the first spinal rod at a first angle and the second spinal rod can extend away from the proximal anchor portion of the second spinal rod at a second angle that is the same or different than the first angle.

FIG. 11 illustrates a prior art insertion tool 500 for implanting spinal rods. Insertion tool is described in U.S. Pat. No. 9,005,205 to Black et al. for ROD INSERTION TOOLS, RODS, AND METHODS. Briefly, insertion tool 500 includes a handle 510 connected to a main body 512. The main body 512 defines an inner passageway 514 that extends between proximal 516 and distal 518 ends of the main body 512. A proximal opening 520 is disposed on the proximal end 516 of the main body 512 and provides communication to the inner passageway 514. Similarly, a distal opening 522 is disposed on the distal end 518 of the main body 512 and provides communication to the inner passageway 514. A rod engaging member (not visible in FIG. 11) is disposed within the inner passageway 514. An adjustment mechanism 526 is operably connected to an end of the rod engaging member and adapted to cause movement of the rod engaging member 524 toward one or both of the proximal 516 and distal 518 ends of the main body 512. A modular arm 550 defines a pair of opposing arms 556, 558 that can be received by complimentary slots 570, 572 defined by the main body 512 of the insertion tool 500. The distal end 552 of the modular arm 550 defines multiple u-shaped channels suitable for contacting and/or interfacing with a tower associated with one or more pedicle screws through which an engaged spinal rod is being passed. Insertion tool provides suitable structure for inserting a spinal rod through pedicle screws that have been previously driven into vertebrae and that are associated with pedicle screw towers in the conventional fashion. While effective, this approach to inserting spinal rods, which utilizes insertion tool 500, requires driving pedicle screws into vertebrae before inserting the spinal rod into the implant site. Moreover, this approach to inserting spinal rods requires the use of pedicle screw towers for positioning the screws and the rod. Indeed, the modular arm 550 of insertion tool 500 has a singular purpose—to space the main body 512, and therefore a spinal rod engaged by the rod engaging member, from the previously-placed pedicle screws by a predetermined distance.

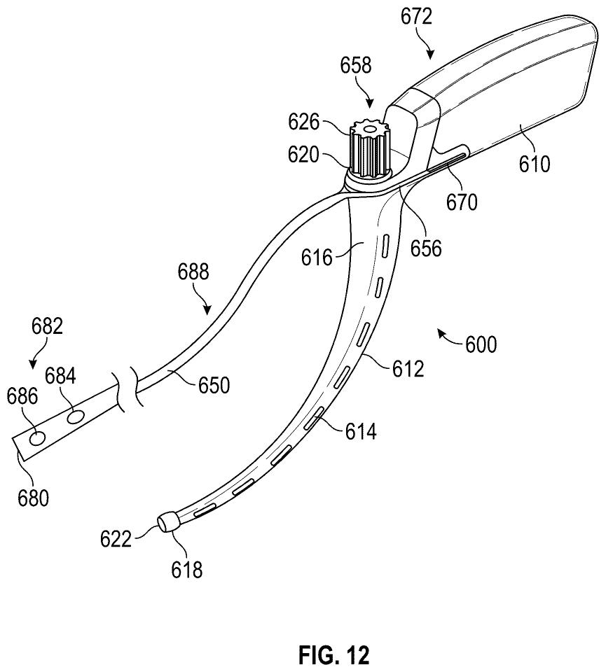

FIG. 12 illustrates an example insertion tool 600 for implanting a spinal rod. Insertion tool 600 is similar to insertion tool 500, except as detailed below. Thus, insertion tool 600 includes a handle 610 connected to a main body 612. The main body 612 defines an inner passageway 614 that extends between proximal 616 and distal 618 ends of the main body 612. A proximal opening 620 is disposed on the proximal end 616 of the main body 612 and provides communication to the inner passageway 614. Similarly, a distal opening 622 is disposed on the distal end 618 of the main body 612 and provides communication to the inner passageway 614. A rod engaging member (not visible in FIG. 12) is disposed within the inner passageway 614. An adjustment mechanism 626 is operably connected to an end of the rod engaging member and adapted to cause movement of the rod engaging member 624 toward one or both of the proximal 616 and distal 618 ends of the main body 612. An arm 650 defines a pair of opposing arms 656, 658 that can be received by complimentary slots 670, 672 defined by the main body 612 of the insertion tool 600.

Insertion tool 600 includes structure that eliminates the need for pedicle screw towers and prepositioning pedicle screws in vertebrae prior to insertion of a spinal rod. Indeed, insertion tool 600 includes structure that enables reversal of the ordering of driving screws and inserting a spinal rod during a spinal rod implantation. As described above, prior art insertion tool 500 includes structure for interacting with pedicle screw towers engaged with previously-placed pedicle screws to insert an engaged spinal rod through the previously-placed pedicle screws. Insertion tool 600, in contrast, includes structure that enables a process in which a spinal rod is inserted into an implant site prior to placement of pedicle screws in the implant site, and eliminates altogether the need for pedicle screw towers. Critical to this is the structure of arm 650, which has a distal end 680 that extends axially beyond the axial position of distal end 618 of the main body 612. Also, arm 650 includes distal portion 682, the entire axial length of which extends axially beyond the axial position of distal end 618 of the main body 612, that defines first 684 and second 686 guide passageways that can be used as guides while driving screws through passageways defined by a spinal rod engaged with insertion tool 600. Optionally, arm 650 can define a curve 688 that positions distal portion 682 out of plane with main body 612. Also critical to performance of insertion tool 600 without pedicle screw towers, the first guide passageway 684 extends through arm 650 at an angle that defines an axial trajectory that matches the axial trajectory of a first passageway of a spinal rod according to an embodiment. Also critical to performance of insertion tool 600, the second guide passageway 686 extends through arm 650 at an angle that defines an axial trajectory that matches the axial trajectory of a second passageway of a spinal rod according to an embodiment. This matching of axial trajectories between passageways of a spinal rod according to an embodiment and guide passageways of an insertion tool according to an embodiment enables insertion of a spinal rod into an implant site, either surgically or percutaneously, and subsequently insertion of screws, such as pedicle screws, through the passageways of the spinal rod while the spinal rod remains engaged with insertion tool 600 using guide passageways 684, 686 of the insertion tool. With passageways in the spinal rod that are based on attributes of the implant site, and guide passageways that match the axial trajectory of the passageways of the spinal rod, insertion tool 600 enables patient-specific screw placement with patient specific spinal rods, while eliminating the need for pedicle screw towers.

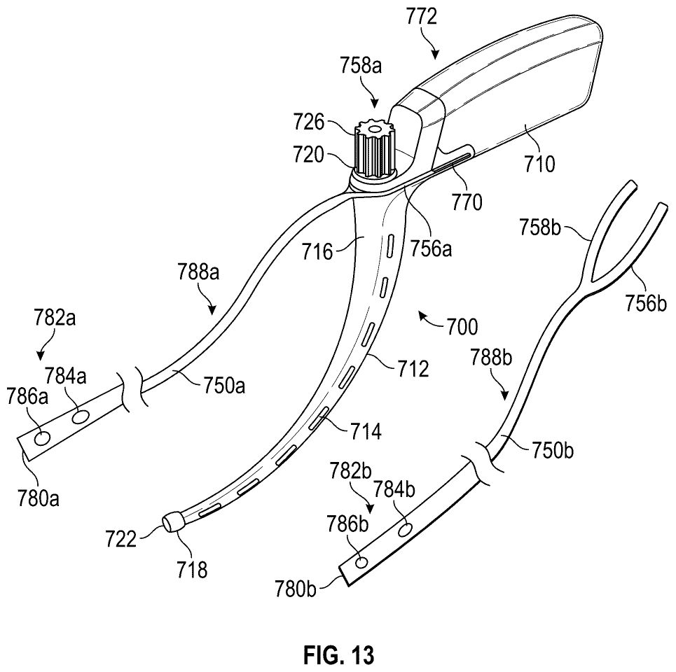

FIG. 13 illustrates another example insertion tool 700 for implanting a spinal rod. Insertion tool 700 is similar to insertion tool 600, except as detailed below. Thus, insertion tool 700 includes a handle 710 connected to a main body 712. The main body 712 defines an inner passageway 714 that extends between proximal 716 and distal 718 ends of the main body 712. A proximal opening 720 is disposed on the proximal end 716 of the main body 712 and provides communication to the inner passageway 714. Similarly, a distal opening 722 is disposed on the distal end 718 of the main body 712 and provides communication to the inner passageway 714. A rod engaging member (not visible in FIG. 13) is disposed within the inner passageway 714. An adjustment mechanism 726 is operably connected to an end of the rod engaging member and adapted to cause movement of the rod engaging member 724 toward one or both of the proximal 716 and distal 718 ends of the main body 712. A first arm 750 defines a pair of opposing arms 756, 758 that can be received by complimentary slots 770, 772 defined by the main body 712 of the insertion tool 700. First arm 750a has a distal end 780a that extends axially beyond the axial position of distal end 718 of the main body 712. Also, first arm 750a includes distal portion 782a, the entire axial length of which extends axially beyond the axial position of distal end 718 of the main body 712, that defines first 784a and second 786a guide passageways that can be used as guides while driving screws through passageways defined by a spinal rod engaged with insertion tool 700. Optionally, first arm 750a can define a curve 788a that positions distal portion 782a out of plane with main body 712. Also critical to performance of insertion tool 700 without pedicle screw towers, the first guide passageway 784a extends through first arm 750a at an angle that defines an axial trajectory that matches the axial trajectory of a first passageway of a spinal rod according to an embodiment. Also critical to performance of insertion tool 700, the second guide passageway 786a extends through first arm 750a at an angle that defines an axial trajectory that matches the axial trajectory of a second passageway of a spinal rod according to an embodiment.

In this embodiment, first arm 750a is modular and can be detached from main body 712 of the insertion tool 700. Insertion tool 700 includes second arm 780a, which is modular and can be attached to main body 712 when first arm 780 is not attached to main body 712. Second arm 780b has a similar structure to first arm 780a except as described below. Thus second arm 780b defines a pair of opposing arms 756b, 758b that can be received by complimentary slots 770, 772 defined by the main body 712 of the insertion tool 700. Second arm 750b has a distal end 780b that extends axially beyond the axial position of distal end 718 of the main body 712. Also, second arm 750b includes distal portion 782b, the entire axial length of which extends axially beyond the axial position of distal end 718 of the main body 712, that defines first 784b and second 786b guide passageways that can be used as guides while driving screws through passageways defined by a spinal rod engaged with insertion tool 700. Optionally, second arm 750b can define a curve 788b that positions distal portion out of plane with main body 712. Also critical to performance of insertion tool 700 without pedicle screw towers, the first guide passageway 784b extends through second arm 750b at an angle that defines an axial trajectory that matches the axial trajectory of a first passageway of a second spinal rod according to an embodiment. Also critical, the second guide passageway 786b extends through second arm 750b at an angle that defines an axial trajectory that matches the axial trajectory of a second passageway of a second spinal rod according to an embodiment.

Each of the first guide passageway 784a and second guide passageway 786a of the first arm 750a defines an axial trajectory that matches an axial trajectory of one passageway of a first spinal rod according to an embodiment, and each of the first guide passageway 784b and second guide passageway 786b of the second arm 750b defines an axial trajectory that matches an axial trajectory of one passageway of a second spinal rod according to an embodiment that is not the first spinal rod. This matching of axial trajectories between passageways of two different spinal rods according to embodiment and guide passageways on two different arms of an insertion tool according to an embodiment enables insertion of two spinal rods into respective implant sites, either surgically or percutaneously, and subsequently insertion of screws, such as pedicle screws, through the passageways of each spinal rod while the respective spinal rod remains engaged with insertion tool using guide passageways of the appropriate arm 750a, 750b of the insertion tool 700. With passageways in the spinal rods that are based on attributes of the respective implant site, and guide passageways that match the axial trajectory of the passageways of the respective spinal rod, insertion tool 700 enables patient-specific screw placement with multiple patient specific spinal rods, while eliminating the need for pedicle screw towers.

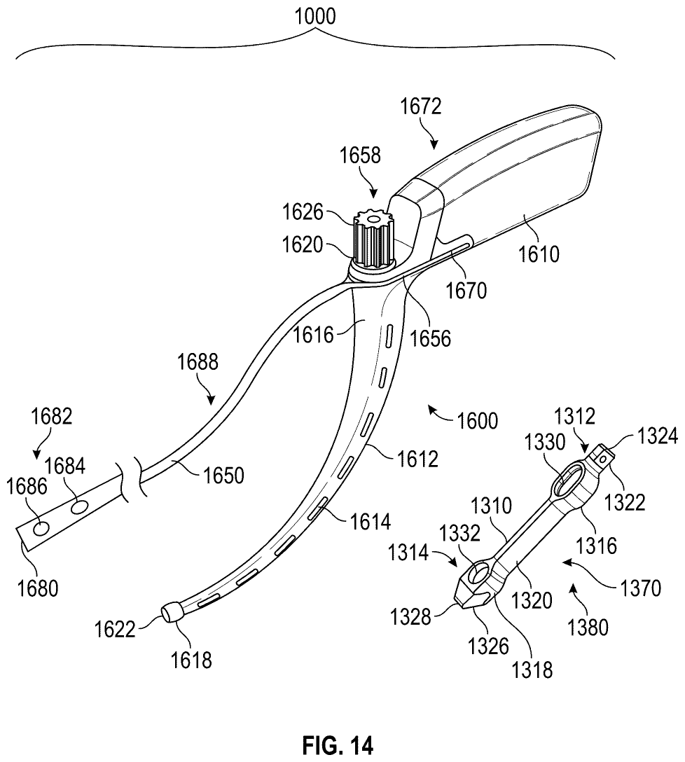

FIG. 14 is a perspective view of an example system 1000 for implanting a spinal rod. System 1000 includes spinal rod 1300 and insertion tool 1600. Spinal rod 1300 is a spinal rod according to an embodiment and insertion tool 1600 is an insertion tool according to an embodiment.

In this example system 1000, spinal rod 1300 is spinal rod 300, described above and illustrated in FIGS. 7, 8, and 9. Thus, spinal rod 1300 is an elongate member 1310 having a proximal end 1312 and a distal end 1314. The elongate member 1310 defines a proximal anchor portion 1316 distal to the proximal end 1312, a distal anchor portion 1318 proximal to the distal end 1314, and a body 1320 extending between the proximal anchor portion 1316 and the distal anchor portion 1318. The proximal end 1312 defines an engagement extension 1322 proximal to the proximal anchor portion 1316 and that defines the terminal proximal surface 1324 of the elongate member 1310. The distal end 1314 defines a nose 1326 distal to the distal anchor portion 1318 and that defines the terminal distal surface 1328 of the elongate member 1310. The proximal anchor portion 1316 defines a passageway 1330 through which an orthopedic screw, such as screw 1302, can be disposed for securing spinal rod 1300 to vertebrae of a patient. Similarly, distal anchor portion 1318 defines a passageway 1332 through which an orthopedic screw, such as screw 1304, can be disposed for securing spinal rod 1300 to vertebrae of a patient. Elongate member 1310 defines curve 1370 along its axial length.

In this example system 1000, insertion tool 1600 is insertion tool 600 described above and illustrated in FIG. 12. Thus, insertion tool 1600 includes a handle 1610 connected to a main body 1612. The main body 1612 defines an inner passageway 1614 that extends between proximal 1616 and distal 1618 ends of the main body 1612. A proximal opening 1620 is disposed on the proximal end 1616 of the main body 1612 and provides communication to the inner passageway 1614. Similarly, a distal opening 1622 is disposed on the distal end 1618 of the main body 1612 and provides communication to the inner passageway 1614. A rod engaging member (not visible in FIG. 14) is disposed within the inner passageway 1614. An adjustment mechanism 1626 is operably connected to an end of the rod engaging member and adapted to cause movement of the rod engaging member 1624 toward one or both of the proximal 1616 and distal 1618 ends of the main body 1612. An arm 1650 defines a pair of opposing arms 1656, 1658 that can be received by complimentary slots 1670, 1672 defined by the main body 1612 of the insertion tool 1600. Arm 1650, which has a distal end 1680 that extends axially beyond the axial position of distal end 1618 of the main body 1612. Also, arm 1650 includes distal portion 1682, the entire axial length of which extends axially beyond the axial position of distal end 1618 of the main body 612, that defines first 1684 and second 1686 guide passageways that can be used as guides while driving screws through passageways defined by spinal rod 1300 when engaged with insertion tool 1600. Optionally, arm 1650 can define a curve 1688 that positions distal portion 1682 out of plane with main body 1612. The first guide passageway 1684 extends through arm 1650 at an angle that defines an axial trajectory that matches the axial trajectory of passageway 1330 of proximal anchor portion 1316 of spinal rod 1300. Also, second guide passageway 1686 extends through arm 1650 at an angle that defines an axial trajectory that matches the axial trajectory of passageway 1332 of distal anchor portion 1318 of spinal rod 1300. This matching of axial trajectories between passageways 1330, 1332 of spinal rod 1300 and guide passageways 1684, 1686 of insertion tool 1600 enables insertion of spinal rod 1300 into an implant site, either surgically or percutaneously, and subsequently insertion of screws, such as pedicle screws, through the passageways 1330, 1332 of the spinal rod 1300 while the spinal rod 1300 remains engaged with insertion tool 1600 using guide passageways 1684, 1686 of the insertion tool 1600. Passageways 1330, 1332 of spinal rod 1300 are based on attributes of a specific implant site in a specific patient. Since guide passageways 1684, 1686 match the axial trajectory of the passageways 1330, 1332 of spinal rod 1300, insertion tool 1600 enables patient-specific screw placement with patient specific spinal rod 1300, while eliminating the need for pedicle screw towers.

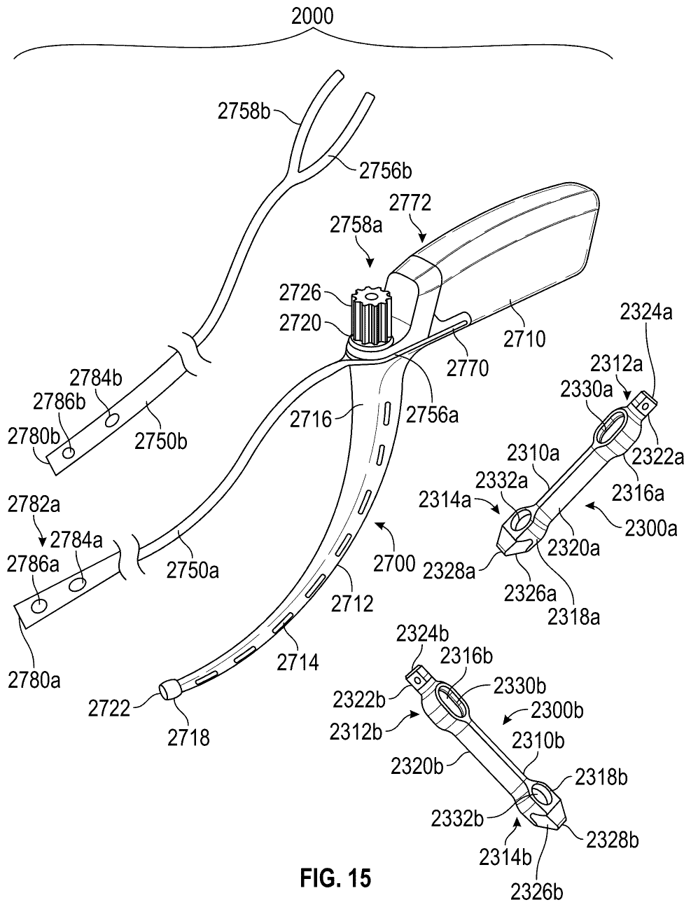

FIG. 15 is a perspective view of another example system 2000 for implanting spinal rods. System 2000 includes first spinal rod 2300a and second spinal rod 2300b, and insertion tool 2700. Each of the first spinal rod 2300a and second spinal rod 2300b is a spinal rod according to an embodiment and insertion tool 2700 is an insertion tool according to an embodiment.

In this example system 2000, each of first spinal rod 2300a and second spinal rod 2300b is spinal rod 300, described above and illustrated in FIGS. 7, 8, and 9. Thus, first spinal rod 2300a is an elongate member 2310a having a proximal end 2312a and a distal end 2314a. The elongate member 2310a defines a proximal anchor portion 2316a distal to the proximal end 2312a, a distal anchor portion 2318a proximal to the distal end 2314a, and a body 2320a extending between the proximal anchor portion 2316a and the distal anchor portion 2318a. The proximal end 2312a defines an engagement extension 2322a proximal to the proximal anchor portion 2316a and that defines the terminal proximal surface 2324a of the elongate member 2310a. The distal end 2314a defines a nose 2326a distal to the distal anchor portion 2318a and that defines the terminal distal surface 2328a of the elongate member 2310a. The proximal anchor portion 2316a defines a passageway 2330a through which an orthopedic screw, such as screw 2302a, can be disposed for securing spinal rod 2300a to vertebrae of a patient. Similarly, distal anchor portion 2318a defines a passageway 2332a through which an orthopedic screw, such as screw 2304a, can be disposed for securing spinal rod 2300a to vertebrae of a patient. Elongate member 2310a defines curve 2370a along its axial length.

Second spinal rod 2300b is an elongate member 2310b having a proximal end 2312b and a distal end 2314b. The elongate member 2310b defines a proximal anchor portion 2316b distal to the proximal end 2312b, a distal anchor portion 2318b proximal to the distal end 2314b, and a body 2320b extending between the proximal anchor portion 2316b and the distal anchor portion 2318b. The proximal end 2312b defines an engagement extension 2322b proximal to the proximal anchor portion 2316a and that defines the terminal proximal surface 2324b of the elongate member 2310a. The distal end 2314b defines a nose 2326b distal to the distal anchor portion 2318b and that defines the terminal distal surface 2328b of the elongate member 2310b. The proximal anchor portion 2316b defines a passageway 2330b through which an orthopedic screw, such as screw 2302b, can be disposed for securing spinal rod 2300b to vertebrae of a patient. Similarly, distal anchor portion 2318b defines a passageway 2332b through which an orthopedic screw, such as screw 2304b, can be disposed for securing spinal rod 2300b to vertebrae of a patient. Elongate member 2310b defines curve 2370b along its axial length.

In this example system 2000, insertion tool 2700 is insertion tool 700 described above and illustrated in FIG. 13. Thus, insertion tool 2700 includes a handle 2710 connected to a main body 2712. The main body 2712 defines an inner passageway 2714 that extends between proximal 2716 and distal 2718 ends of the main body 2712. A proximal opening 2720 is disposed on the proximal end 2716 of the main body 2712 and provides communication to the inner passageway 2714. Similarly, a distal opening 2722 is disposed on the distal end 2718 of the main body 2712 and provides communication to the inner passageway 2714. A rod engaging member (not visible in FIG. 15) is disposed within the inner passageway 2714. An adjustment mechanism 2726 is operably connected to an end of the rod engaging member and adapted to cause movement of the rod engaging member 2724 toward one or both of the proximal 2716 and distal 2718 ends of the main body 2712. A first arm 2750a defines a pair of opposing arms 2756a, 2758a that can be received by complimentary slots 2770, 2772 defined by the main body 2712 of the insertion tool 2700. First arm 2750a has a distal end 2780a that extends axially beyond the axial position of distal end 2718 of the main body 2712. Also, first arm 2750a includes distal portion 2782a, the entire axial length of which extends axially beyond the axial position of distal end 2718 of the main body 2712, that defines first 2784a and second 2786a guide passageways that can be used as guides while driving screws through passageways defined by first spinal rod 2300a when engaged with insertion tool 2700. First guide passageway 2784a extends through first arm 2750a at an angle that defines an axial trajectory that matches the axial trajectory of the passageway 2330a defined by the proximal anchor portion 2316 of the first spinal rod 2300a. The second guide passageway 2786a extends through first arm 2750a at an angle that defines an axial trajectory that matches the axial trajectory of the passageway 2332a defined by the distal anchor portion 2318 of first spinal rod 2300a. First arm 2750a is modular and can be detached from main body 2712 of the insertion tool 2700. Insertion tool 2700 includes second arm 2780a, which is modular and can be attached to main body 2712 when first arm 2780a is not attached to main body 2712. Second arm 2780b has a similar structure to first arm 2780a except as described below. Thus second arm 2780b defines a pair of opposing arms 2756b, 2758b that can be received by complimentary slots 2770, 2772 defined by the main body 2712 of the insertion tool 2700. Second arm 2750b has a distal end 2780b that extends axially beyond the axial position of distal end 2718 of the main body 2712 when second arm is attached to main body 2712. Also, second arm 2750b includes distal portion 2782b, the entire axial length of which extends axially beyond the axial position of distal end 2718 of the main body 2712, that defines first 2784b and second 2786b guide passageways that can be used as guides while driving screws through passageways defined by the second spinal rod 2300b when engaged with insertion tool 2700. Also critical to performance of insertion tool 700 without pedicle screw towers, the first guide passageway 2784b extends through second arm 2750b at an angle that defines an axial trajectory that matches the axial trajectory of passageway 2330b defined by the proximal anchor portion 2316b of the second spinal rod 2300b. Also critical to performance of insertion tool 700 without pedicle screw towers, the second guide passageway 2786b extends through second arm 2750b at an angle that defines an axial trajectory that matches the axial trajectory of passageway 2332b defined by the distal anchor portion 2318b of second spinal rod 2300b.

This matching of axial trajectories between passageways of two different spinal rods 2300a, 2300b and guide passageways on two different arms 2750a, 2750b of insertion tool 2700 enables insertion spinal rods 2300a, 2300b into respective implant sites, either surgically or percutaneously, and subsequent insertion of screws, such as pedicle screws, through the passageways 2330a, 2332a, 2330b, 2332b of the spinal rods 2300a, 2300b while the respective spinal rod 2300a, 2300b remains engaged with insertion tool 2700 using guide passageways 2784a, 27861, 2784b, 2786b of the appropriate arm 2750a, 2750b of the insertion tool 2700. With passageways 2330a, 2332a, 2330b, 2332b in the spinal rods 2300a, 2300b that are based on attributes of the respective implant site, and guide passageways 2784a, 27861, 2784b, 2786b that match the axial trajectory of the passageways of the respective spinal rod, system 2000 enables patient-specific screw placement with multiple patient specific spinal rods while eliminating the need for pedicle screw towers.

In all spinal rod embodiments, screws are not considered an element of the spinal rod. Screws are illustrated in the various figures of example spinal rod embodiments for reference and convenience. In all system embodiments, screws are considered optional elements of the system for implanting spinal rods. Inclusion of screws in systems according to embodiments is considered advantageous, though, at least because their inclusion enables efficient use of the system and provides a level of assurance of fit and compatibility with the spinal rods of the system.

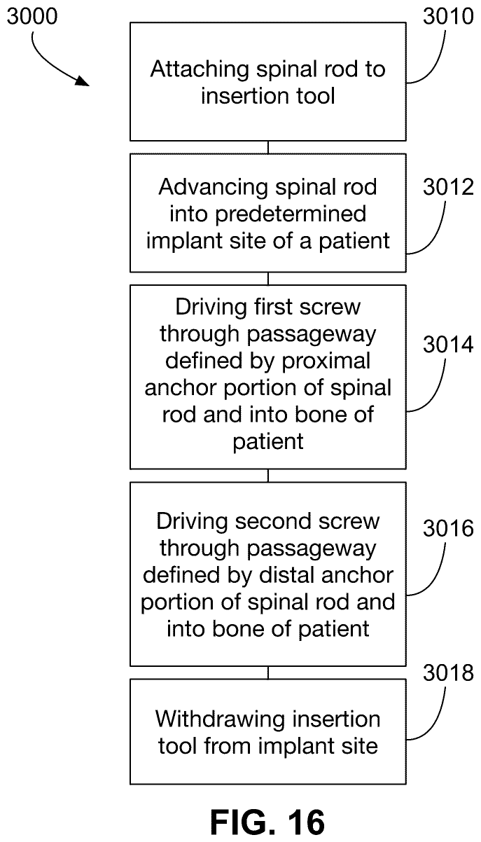

FIG. 16 is a flowchart illustration of an example method 3000 of implanting a spinal rod. The method includes use of an insertion tool according to an embodiment to implant a spinal rod according to an embodiment. An initial step 3010 comprises attaching a spinal rod according to an embodiment to an insertion tool according to an embodiment. Another step 3012 comprises advancing the spinal rod into a predetermined implant site of a patient. Another step 3014 comprises driving a first screw through a passageway defined by a proximal anchor portion of the spinal rod and into a bone of the patient by extending a driving tool through a passageway defined by the arm of the insertion tool that has the same axial trajectory of the passageway defined by a proximal anchor portion of the spinal rod. Another step 3016 comprises driving a second screw through a passageway defined by a distal anchor portion of the spinal rod and into a bone of the patient by extending a driving tool through a passageway defined by the arm of the insertion tool that has the same axial trajectory of the passageway defined by a distal anchor portion of the spinal rod and into a bone of the patient, which can be the same or a different bone than the bone in step 3014. Another step 3018 comprises withdrawing the insertion tool from the implant site. Completion of step 3018 results in the spinal rod being implanted in the implant site of the patient. Any of steps 3012, 3014, and 3016 can be performed with augmented reality equipment and techniques, such as augmented reality navigation known in the art.

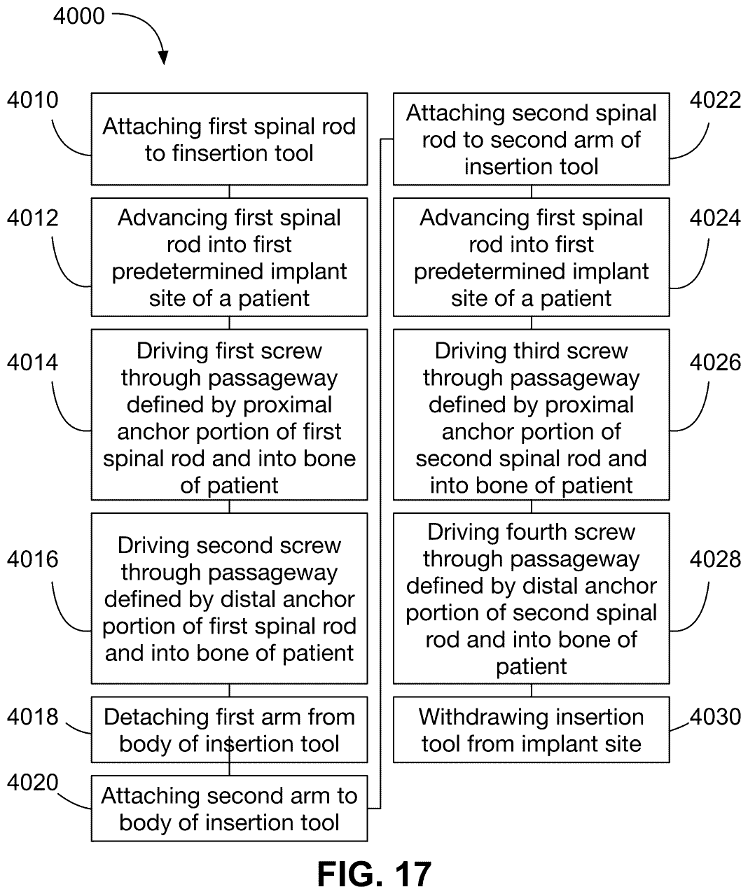

FIG. 17 is a flowchart illustration of an example method 4000 of implanting spinal rods. The method includes use of a system for implanting spinal rods according to an embodiment. An initial step 4010 comprises attaching a first spinal rod of a system according to an embodiment to an insertion tool of a system according to an embodiment. For this step, the insertion tool has a first arm attached to the body, with guide passageways having axial trajectories that match respective anchor portion passageways of the first spinal rod. Another step 4012 comprises advancing the first spinal rod into a first predetermined implant site of a patient. Another step 4014 comprises driving a first screw through a passageway defined by a proximal anchor portion of the first spinal rod and into a bone of the patient by extending a driving tool through a passageway defined by the arm of the insertion tool that has the same axial trajectory of the passageway defined by a proximal anchor portion of the first spinal rod. Another step 4016 comprises driving a second screw through a passageway defined by a distal anchor portion of the first spinal rod and into a bone of the patient, which can be the same or a different bone than the bone in step 4014, by extending a driving tool through a passageway defined by the arm of the insertion tool that has the same axial trajectory of the passageway defined by a distal anchor portion of the first spinal rod. At this point, an optional step of withdrawing the insertion tool from the first implant site can be performed prior to initiating one or both of step 4018 and step 4020. Another step 4018 comprises detaching the first arm from the body of the insertion tool. Another step 4020 comprises attaching a second arm to the body of the insertion tool. The second arm has guide passageways having axial trajectories that match respective anchor portion passageways of the second spinal rod. Accordingly, the second arm advantageously has guide passageways having axial trajectories that are different from the axial trajectories of the guide passageways defined by the first arm. It is noted, though, that the second arm can have guide passageways having axial trajectories that are the same as the axial trajectories of the guide passageways defined by the first arm. Another step 4022 comprises attaching the second spinal rod of the system to the insertion tool. Another step 4024 comprises advancing the second spinal rod into a predetermined implant site of a patient that is different from the first predetermined implant site. Another step 4026 comprises driving a third screw through a passageway defined by a proximal anchor portion of the second spinal rod and into a bone of the patient, which is advantageously a different bone than the bone or bones into which the first and second screws have been driven into, by extending a driving tool through a passageway defined by the arm of the insertion tool that has the same axial trajectory of the passageway defined by a proximal anchor portion of the second spinal rod. Another step 4028 comprises driving a fourth screw through a passageway defined by a distal anchor portion of the second spinal rod and into a bone of the patient, which can be the same or a different bone than the bone in step 4026 by extending a driving tool through a passageway defined by the arm of the insertion tool that has the same axial trajectory of the passageway defined by a distal anchor portion of the second spinal rod. Another step 4030 comprises withdrawing the insertion tool from the implantation site. Completion of step 4030 results in the first and second spinal rods being implanted in the first and second implant sites of the patient, respectively. Any of steps 4012, 4014, 4016, 4024, 4026, and 4028 can be performed with augmented reality equipment and techniques, such as augmented reality navigation known in the art.

The disclosure also relates to methods of making spinal rods, methods of making sets of spinal rods, methods of making insertion tools, and methods of making systems for implanting a spinal rod in a patient. Methods of making include a step of obtaining one or more attributes of a specific implant site, such as one or more pre-operative images of an intended implantation site in a specific patient, and at least one of the following steps: making a spinal rod having a structural property based on the one or more attributes of a specific implant site, making multiples spinal rods, each of the multiple spinal rods having a structural property based on the one or more attributes of a specific implant site, making an arm having at least one passageway matching an axial trajectory of a passageway of a specific spinal rod according to a particular embodiment, and making multiple arms, each of the multiple arms having at least one passageway matching an axial trajectory of a passageway of a specific spinal rod according to a particular embodiment.

Those with ordinary skill in the art will appreciate that various modifications and alternatives for the described and illustrated examples can be developed in light of the overall teachings of the disclosure, and that the various elements and features of one example described and illustrated herein can be combined with various elements and features of another example without departing from the scope of the invention. Accordingly, the particular arrangements of elements and steps disclosed herein have been selected by the inventor simply to describe and illustrate examples of the invention and are not intended to limit the scope of the invention or its protection, which is to be given the full breadth of the appended claims and any and all equivalents thereof.

What is claimed is:

1. A method of implanting spinal rods in a patient, the method comprising:
attaching a first spinal rod to an insertion tool having a first insertion tool body and a first arm attached to the first insertion tool body and defining a first guide passageway having a first guide passageway axial trajectory and a second guide passageway having a second guide passageway axial trajectory, the first spinal rod defining a first proximal anchor portion defining a first proximal anchor portion passageway having a first proximal anchor portion passageway trajectory matching the first guide passageway axial trajectory and a first distal anchor portion defining a first distal anchor portion passageway having a first distal anchor portion passageway trajectory matching the second guide passageway axial trajectory;
advancing the first spinal rod into a first predetermined implant site of the patient;
driving a first screw through the first proximal anchor portion passageway and into a bone of the patient by extending a driving tool through the first guide passageway to engage the first screw;
driving a second screw through the first distal anchor portion passageway and into a bone of the patient by extending a driving tool through the second guide passageway to engage the second screw;
detaching the first arm from the insertion tool body;
attaching a second arm to the insertion tool body, the second arm defining a third guide passageway having a third guide passageway axial trajectory and a fourth guide passageway having a fourth guide passageway axial trajectory;
attaching a second spinal rod to the insertion tool, the second spinal rod defining a second proximal anchor portion defining a second proximal anchor portion passageway having a second proximal anchor portion passageway trajectory matching the third guide passageway axial trajectory and a second distal anchor portion defining a second distal anchor portion passageway having a second distal anchor portion passageway trajectory matching the fourth guide passageway axial trajectory;
advancing the second spinal rod into a second predetermined implant site of the patient
driving a third screw through the second proximal anchor portion passageway and into a bone of the patient by extending a driving tool through the third guide passageway to engage the second screw;
driving a fourth screw through the second distal anchor portion passageway and into a bone of the patient by extending a driving tool through the fourth guide passageway to engage the second screw; and
withdrawing the insertion tool from the implantation site.

2. The method of claim 1, further comprising withdrawing the insertion tool from the first implant site before initiating the detaching the first arm from the insertion tool body and attaching a second arm to the insertion tool body.

3. The method of claim 1, wherein the driving a first screw through the first proximal anchor portion passageway and into a bone of the patient and the driving a second screw through the first distal anchor portion passageway and into a bone of the patient are performed so that the first and second screws are driven into the same bone.

4. The method of claim 1, wherein the driving a first screw through the first proximal anchor portion passageway and into a bone of the patient and the driving a second screw through the first distal anchor portion passageway and into a bone of the patient are performed so that the first and second screws are driven into different bones.

5. The method of claim 4, wherein the driving a third screw through the second proximal anchor portion passageway and into a bone of the patient and the driving a fourth screw through the second proximal anchor portion passageway and into a bone of the patient are performed so that the third and fourth screws are driven into the same bone.

6. The method of claim 4, wherein the driving a third screw through the second proximal anchor portion passageway and into a bone of the patient and the driving a fourth screw through the second proximal anchor portion passageway and into a bone of the patient are performed so that the third and fourth screws are driven into different bones.

7. The method of claim 1, first proximal anchor portion passageway trajectory and the first distal anchor portion passageway trajectory are the same.

8. The method of claim 1, wherein the first proximal anchor portion passageway trajectory and the first distal anchor portion passageway trajectory are different.

9. The method of claim 1, wherein the third guide passageway axial trajectory and the first guide passageway axial trajectory are different.

10. The method of claim 9, wherein the fourth guide passageway axial trajectory and the second guide passageway axial trajectory are different.

11. The method of claim 1, wherein the first proximal anchor portion passageway trajectory, first distal anchor portion passageway trajectory, second proximal anchor portion passageway trajectory, and second distal anchor portion passageway trajectory are different.

12. The method of claim 1, wherein the first spinal rod defines a first width at a first location along the axial length of the body and a second width at a second location along the axial length of the body; and
wherein the first and second widths are different.

13. The method of claim 12, wherein the second spinal rod defines a third width at a third location along the axial length of the body and a fourth width at a fourth location along the axial length of the body; and
wherein the third and fourth widths are different.

14. The method of claim 13, wherein the first, second, third, and fourth widths are different.

15. The method of claim 1, wherein the first arm defines a first tab and the second arm defines a second tab; and
Wherein the body of the insertion tool defines a slot configured to selectively receive the first and second tabs.

16. The method of claim 1, wherein the first spinal rod includes one or more structural features that are based upon one or more dimensions calculated based on pre-operative imaging of the implant site of the patient.

17. The method of claim 16, wherein the one or more structural features comprises one or more of the first proximal anchor portion passageway trajectory and the first distal anchor portion passageway trajectory.

18. The method of claim 16, wherein the one or more structural features comprises the first proximal anchor portion passageway trajectory and the first distal anchor portion passageway trajectory.

19. The method of claim 18, wherein the one or more dimensions comprise a distance between bones.

20. The method of claim 19, wherein the first spinal rod includes a cross-sectional shape of the body, a width of the body, or an outer diameter of the body that is based upon the body mass index (BMI) of the patient.
